(19) United States
(12) Patent Application Publication
(10) Pub. No.: US 2026/0165854 A1
(43) Pub. Date: Jun. 18, 2026

(54) ANATOMICALLY ALIGNED PROSTHETIC FOOT

(71) Applicant: LOMA LINDA UNIVERSITY, Loma Linda, CA (US)

(72) Inventors: Michael Davidson, Loma Linda, CA (US); John Wetteland, Loma Linda, CA (US); Ethan Davidson, Loma Linda, CA (US)

(73) Assignee: LOMA LINDA UNIVERSITY, Loma Linda, CA (US)

(21) Appl. No.: 19/126,751

(22) PCT Filed: Nov. 2, 2023
(86) PCT No.: PCT/US2023/078559

§ 371 Date: May 2, 2025

Related U.S. Application Data
(60) Provisional application No. 63/382,072, filed on Nov. 2, 2022.

(51) Int. Cl. A61F 2/66; A61F 2/50

(52) U.S. Cl. CPC A61F 2/6607; A61F 2002/5006; A61F 2002/5018

(57) ABSTRACT

A prosthetic limb has transverse planes and sagittal planes parallel to a horizontal and a medial plane of a user, respectively. The prosthetic limb includes a tibia section, an ankle section, and a foot section. The ankle section defines an ankle axis and is rotatably coupled to the tibia section such that the ankle section and the tibia section rotate about the ankle axis. The foot section secures to the ankle section and has a foot sagittal plane defining a line of progression. The foot section includes a base foot and a toe portion pivotally coupled to the base foot such that the toe portion is configured to pivot about a toe axis relative to the base foot. The toe axis is angled relative to the line of progression at a toe angle in a transverse plane of the foot section. The toe angle is less than 90 degrees.

### BACKGROUND

### 1. Technical Field

The present disclosure relates to prosthetic limbs, and more specifically, anatomically aligned prosthetic ankles with passive assist mechanisms.

### 2. Discussion of Related Art

Prosthetic limbs have been used as a replacement for amputated limbs to aid people with an amputation recover at least some degree of movement lost as a result of amputation. While improvements in prosthetic limbs have been made over the years, a persistent problem with many prosthetic limbs is an inability to closely replicate a person's natural or normal (pre-loss) movement. For example, the human ankle serves as an important component for walking, navigating slopes, and even simpler tasks such as sitting, squatting, and standing. In particular, ankle movement is important for providing a push-off force in a pre-swing phase of a person's gait cycle as well as their balance during locomotion, particularly when moving over uneven ground or up a slope, ramp, or stairs. However, even with more advanced and sophisticated prosthetics, the loss of an ankle to amputation continues to present difficulties in walking, balance, and navigation of inclines and declines. These difficulties increase the potential for falling and prose safety risks to people with an amputation. Often, people with an amputation must learn to walk in a different manner and, in effect, retrain their brains to initially recognize their limb/prosthetic foot is missing and to take into account the prosthesis.

### SUMMARY

In an aspect of the present disclosure, a prosthetic limb has transverse planes parallel to a horizontal plane of a user and sagittal planes parallel to a medial plane of the user. The prosthetic limb includes a tibia section, an ankle section, and a foot section. The ankle section defines an ankle axis. The ankle section rotatably couples to the tibia section and is configured to rotate relative to the tibia section about the ankle axis. The foot section secures to the ankle section. The foot section has a foot sagittal plane defining a line of progression of the foot section. The foot section includes a base foot and a toe portion pivotally coupled to the base foot. The toe portion is configured to pivot about a toe axis relative to the base foot. The toe axis is angled relative to the line of progression at a toe angle in a transverse plane of the foot section. The toe angle is less than 90 degrees.

In aspects, the toe angle is between 50 degrees and 80 degrees. The toe angle may be 70 degrees.

In some aspects, the tibia section and the ankle section are configured to be the tibia section and the ankle section of a right prosthetic limb or a left prosthetic limb. The tibia section and the ankle section may be configured to switch between the tibia section and the ankle section of a right prosthetic limb and a left prosthetic limb by rotating the tibia section and the ankle section 180-degrees about a central longitudinal axis thereof.

In certain aspects, the foot section has a foot center line bisecting the foot section longitudinally. The foot center line may be off-set relative to the ankle axis at a foot angle in the transverse plane of the foot section. The foot angle may be in a range of 50 degrees to 100 degrees.

In particular aspects, the prosthetic limb includes a toe flexion member secured to the base foot and the toe portion. The toe flexion member may be configured to assist pivoting of the toe portion relative to the base foot. The toe flexion member may include an adjustment device configured to increase or decrease an assisting force applied by the toe flexion member to assist pivoting of the toe portion relative to the base foot. The toe flexion member may have a first end secured to the base foot and a second end, opposite the first end, secured to the toe portion. The toe flexion member may define a central axis and may be secured to the base foot and the toe portion such that the central axis is substantially perpendicular to the toe axis. The toe flexion member may include a hydropneumatics damper.

In aspects, the toe portion has an inferior side and the base foot has an inferior side. The toe portion may be configured to pivot relative to the base foot between a toe neutral position in which the inferior side of the toe portion is substantially coplanar to the inferior side of the base foot and a toe flexion position in which the inferior side of the toe portion is pivoted away from coplanarity with the inferior side of the base foot. The prosthetic limb may include a toe flexion member configured to apply a push-off force to a ground surface as the toe portion pivots from the toe flexion position to the toe neutral position to assist a user in locomotion. The toe flexion member may include an adjustment device configured to increase or decrease the push-off force applied by the toe flexion member. The toe flexion member may define a central axis and may be secured to the base foot and the toe portion such that the central axis is substantially parallel to the inferior side of the base foot when the toe portion is in the toe neutral position and when the toe portion is in the toe flexion position. The foot section may include a foot connector attached to a superior side of the base foot configured to operably secure the prosthetic limb to a user.

In another aspect of the present disclosure, a prosthetic limb has transverse planes parallel to a horizontal plane of a user, frontal planes parallel to a coronal plane of the user, and sagittal planes parallel to a medial plane of the use. The prosthetic limb includes a tibia section, an ankle section, and a foot section. The ankle section defines an ankle axis that is angled relative to the transverse plane at a non-zero ankle angle in a frontal plane of the ankle section. The ankle section rotatably couples to the tibia section and is configured to rotate relative to the tibia section about the ankle axis. The foot section is secured to the ankle section. The foot section has a foot center line and a foot sagittal plane. The foot center line longitudinally bisects the foot section. The foot section sagittal plane defines a line of progression of the foot section. The foot center line is off-set relative to the line of progression at a non-zero angle in a transverse plane of the foot section. The foot section includes a base foot and a toe portion.

In aspects, the toe portion is pivotally coupled to the base foot and is configured to pivot relative to the base foot. The progression angle may be in the range of 4 degrees to 12 degrees. The progression angle may be 8 degrees. The ankle angle may be in the range of 4 degrees to 12 degrees. The ankle angle may be 8 degrees.

In some aspects, the tibia section has an anterior side and the foot section has a superior side. The ankle section and the tibia section may be rotatable relative to each other about the ankle axis between a neutral position in which the anterior side of the tibia section is substantially perpendicular to the superior side of the foot section, a dorsi-flexion position in which an angle between the anterior side of the tibia section and the superior side of the foot section is decreased, and a plantar-flexion position in which the angle between the anterior side of the tibia section and the superior side of the foot section is increased. The prosthetic limb may include a flexion mechanism configured to passively assist in relative rotation between the tibia section and the ankle section, and pivoting of the toe portion relative to the base foot. The flexion mechanism may include a dorsi-flexion member configured to assist rotation of the tibia section and the ankle section between the neutral position and the dorsi-flexion position. The flexion mechanism may include a plantar-flexion member configured to assist rotation of the tibia section and the ankle section between the neutral position and the plantar-flexion position. The flexion mechanism may include a toe flexion member configured to assist pivoting of the portion relative to the base foot.

In certain aspects, the prosthetic limb includes a flexion mechanism configured to assist relative rotation of the tibia section and the ankle section, and pivoting of the toe portion relative to the foot base. The flexion mechanism may be configured to assist relative rotation of the tibia section and the ankle section, and pivoting of the toe portion relative to the base foot independently of one another. The tibia section and the ankle section may be configured to be the tibia section and the ankle section of a right prosthetic limb or a left prosthetic limb.

In another aspect of the present disclosure, a prosthetic limb has transverse planes parallel to a horizontal plane of a user and frontal planes parallel to a coronal plane of the user. The prosthetic limb includes a tibia section and an ankle section. The ankle section rotatably couples to the tibia section. The ankle section and the tibia section are rotatable about an ankle axis that is angled relative to a transverse plane of the ankle section in a frontal plane of the ankle section. The ankle angle is a non-zero angle. The tibia section and the ankle section are switchable between a left prosthetic limb configuration and a right prosthetic limb configuration by rotating the tibia section and the ankle section 180-degrees about a central longitudinal axis thereof.

In aspects, the ankle angle is in the range of 4 degrees to 12 degrees. The ankle angle may be 8 degrees.

In some aspects, the prosthetic limb includes a dorsi-flexion member configured to assist relative rotation between the tibia section and the ankle section. The dorsi-flexion member may be switchable between a right prosthetic limb configuration and a left prosthetic limb configuration by securing the dorsi-flexion member to a posterior side of the respective right prosthetic limb configuration of the tibia section and the ankle section or the left prosthetic limb configuration of the tibia section and the ankle section. The prosthetic limb may include a plantar-flexion member configured to assist relative rotation between the tibia section and the ankle section. The plantar-flexion member may be switchable between a right prosthetic limb configuration and a left prosthetic limb configuration by securing the plantar-flexion member to an anterior side of the respective right prosthetic limb configuration of the tibia section and the ankle section or the left prosthetic limb configuration of the tibia section and the ankle section.

In another aspect of the present disclosure, a method of assembling a prosthetic limb having transverse planes parallel to a horizontal plane of a user, frontal planes parallel to a coronal plane of the user, and sagittal planes parallel to a medial plane of the user includes orientating a tibia section and an ankle section of the prosthetic limb, and securing a right foot section or a left foot section. The tibia section and the ankle section are oriented in one of a right prosthetic limb configuration or a left prosthetic limb configuration with the tibia section and the ankle section rotatable relative to each other about an ankle axis. The ankle axis is offset relative to the transverse plane at a non-zero ankle angle in a frontal plane of the ankle section. The right foot section or left foot section is secured to the ankle section based on the orientation of the tibia section and the ankle section.

In aspects, the method includes securing a dorsi-flexion member and plantar-flexion member to the tibia section and the ankle section such that the dorsi-flexion member and the plantar-flexion member passively assist relative rotation between the tibia section and the ankle section. The method may include adjusting the dorsi-flexion member or the plantar-flexion member to increase or decrease a force assisting relative rotation between the tibia section and the ankle section. The method may include adjusting a toe flexion member of the right foot section or the left foot section to increase or decrease a force assisting pivoting of a toe portion of the right foot section or of the left foot section.

In certain aspects, the method includes switching the prosthetic limb to the other of the right prosthetic limb configuration or the left prosthetic limb configuration by rotating the tibia section and the ankle section 180-degrees about a central longitudinal axis thereof. The method may include removing a foot section from the ankle section, reorientating the tibia section and the ankle section from one of the right prosthetic limb configuration or the left prosthetic limb configuration to the other of the right prosthetic limb configuration or the left prosthetic limb configuration, and attaching another foot section matching the reorientated tibia section and ankle section.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is a perspective view of a human body illustrating anatomical planes and directions;

FIG. 2 is a perspective view of an anatomically correct foot illustrating the anatomical planes of FIG. 1 with respect thereto and rotational axes thereof;

FIG. 3 is a top view of an anatomically correct foot illustrating a metatarsophalangeal joint axis and a line of progression;

FIG. 4 is a perspective view of an anatomically aligned prosthetic ankle in accordance with the present disclosure;

FIG. 5 is a side view of the prosthetic ankle of FIG. 4;

FIG. 6 is a rear view of the prosthetic ankle of FIG. 4;

FIG. 7 is a top view of the prosthetic ankle of FIG. 4;

FIG. 8 is a side view of another anatomically aligned prosthetic ankle in accordance with the present disclosure in a neutral position thereof;

FIG. 9 is a side view of the prosthetic ankle of FIG. 8 in a plantar-flexion position thereof;

FIG. 10 is a side view of the prosthetic ankle of FIG. 8 in a dorsi-flexion position thereof;

FIG. 11 is a perspective view of another anatomically aligned prosthetic ankle in accordance with the present disclosure in a right ankle configuration thereof;

FIG. 12 is a partial exploded view of the prosthetic ankle of FIG. 11;

FIG. 13 is top view of the prosthetic ankle of FIG. 11;

FIG. 14 is a rear view of the prosthetic ankle of FIG. 11;

FIG. 15 is a perspective view of a tibia section and an ankle section of the prosthetic ankle of FIG. 11;

FIG. 16 is a front view of the tibia section and the ankle section of FIG. 15;

FIG. 17 is a top view of the tibia section and the ankle section of FIG. 15;

FIG. 18 is a perspective view a foot of the prosthetic ankle of FIG. 11;

FIG. 19 is a top view of the foot of FIG. 18;

FIG. 20 is a side view of the prosthetic ankle of FIG. 11 in a toe neutral position thereof;

FIG. 21 is a side view of the prosthetic ankle of FIG. 11 in a toe flexion position thereof;

FIG. 22 is a perspective view of a plantar-flexion member of the prosthetic ankle of FIG. 11;

FIG. 23 is an exploded view of a double rocker mount of the plantar-flexion mount of FIG. 22;

FIG. 24 is a perspective view of a dorsi-flexion member of the prosthetic ankle of FIG. 11;

FIG. 25 is an exploded view of a single rocker mount of the dorsi-flexion member of FIG. 24;

FIG. 26 is a perspective view of a toe flexion member of the prosthetic ankle of FIG. 11;

FIG. 27 is a rear view of an anatomically aligned prosthetic ankle in accordance with the present disclosure in a left ankle configuration thereof;

FIG. 28 is a top view of the prosthetic ankle of FIG. 27; and

FIG. 29 is a flowchart illustrating a method of assembling a prosthetic ankle in accordance with the present disclosure.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

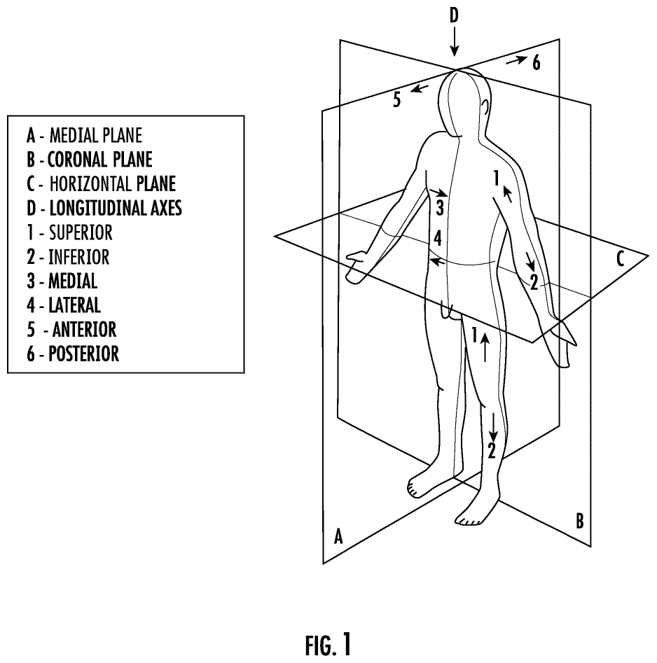

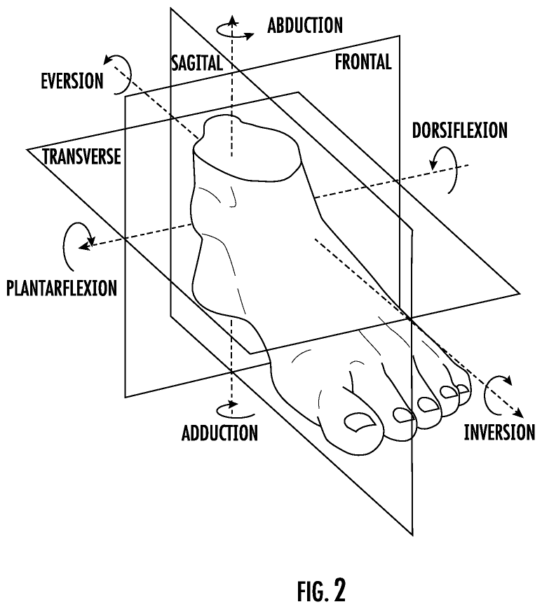

Referring to FIGS. 1-3, as used herein, the term “user” refers to a person with an amputation utilizing a prosthetic or device. Throughout this description, standard anatomical planes and directional terms are used herein to promote clarity of the disclosure and are not intended to be limiting. “Anterior” generally refers to the front side of the device or the user. “Posterior” generally refers to the rear of the device or the user. “Superior” generally means towards the top of the device or the head of the user. “Inferior” generally means towards the bottom of the device or the away from the head of the user. “Medial” generally means towards the midline of the body of the user. “Lateral” generally means away from the midline of the body of the user. “Frontal plane” refers to a vertical plane running from side-to-side that divides any part of the body, e.g., a foot or ankle, into anterior and posterior portions. “Coronal plane” refers to the frontal plane through the midline of the body. “Sagittal plane” refers to a vertical plane running from front to back that divides any part of the body, e.g., a foot or ankle, into right and left sides. “Medial plane” refers to a sagittal plane through the midline of the body. “Horizontal plane” refers to the transverse plane running through the midline of the body and divides the body into upper and lower parts. “Transverse plane” refers to a horizontal plane that divides any part of the body, e.g., a foot or ankle, into upper and lower parts. “Metatarsophalangeal joint axis” (MPT joint axis) refers to a line extending between the MPT joint on the second phalange and the fifth phalange. “Line of Progression” refers to a direction of movement of a user when a user is moving in a substantially forward direction such that the user is moving in a direction orthogonal to the coronal plane. The Line of Progression is parallel to a sagittal plane and the line of progression of a foot is parallel to the sagittal plane of the foot. “Proximal” refers to the portion of the device or component thereof that is closer to the residual limb of a user. “Distal” refers to the portion of the device or component thereof that is farther from the residual limb of a user.

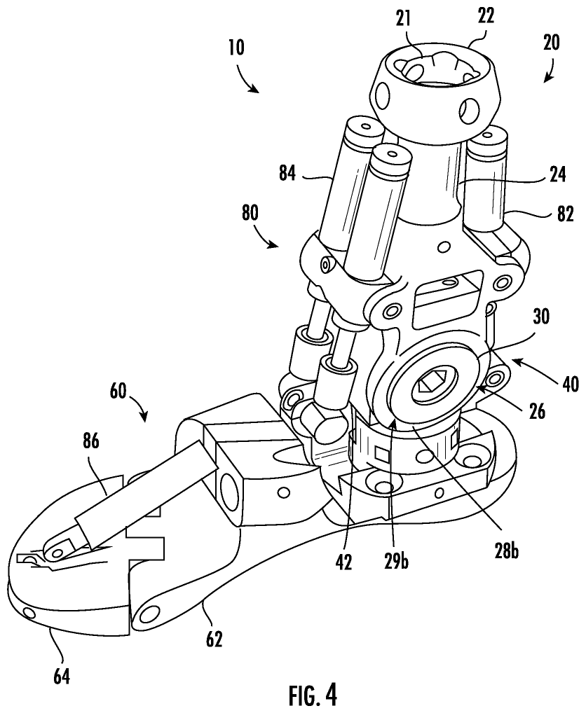

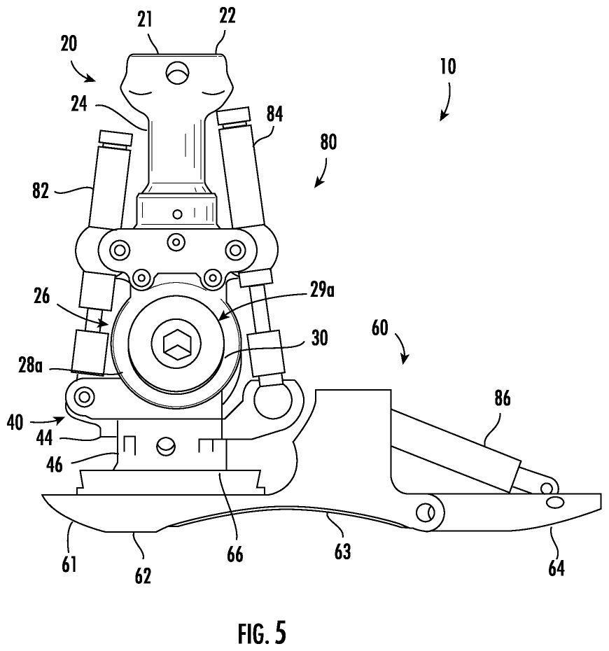

Referring now to FIGS. 4-7, an anatomically aligned prosthetic ankle 10 is provided in accordance with embodiments of the present disclosure. The prosthetic ankle 10 includes a tibia section 20, an ankle section 40, and a foot section 60. The prosthetic ankle 10 includes a flexion mechanism 80 that passively assists movement of the tibia section 20, the ankle section 40, and the foot section 60 relative to one another. The tibia section 20, the ankle section 40, and the foot section 60 move relative to each other about axes based on the rotational axes of an actual ankle or foot. The axes to which the tibia section 20, the ankle section 40, and/or the foot section 60 move about may be offset from the anatomical planes of the body to match the rotational axes of an actual ankle or foot. For example, the tibia section 20 may extend from the ankle section 40 askew at an angle from parallel to the longitudinal axis in the frontal plane. The angle of the tibia section 20 from parallel to the longitudinal axis may be in a range of 4 degrees to 12 degrees, e.g., 8 degrees.

The tibia section 20 includes a tibia proximal end portion 22, a shaft 24, and tibia distal end portion 26. The tibia proximal end portion 22 connects the prosthetic ankle 10 to the user. The tibia proximal end portion 22 has a proximal connector 21. In embodiments, the proximal connector 21 connects to a prosthetic socket (not shown) which receives the residual limb of a user. In some embodiments, the proximal connector 21 connects to a prosthetic knee (not shown). For example, the proximal connector 21 may connect to a tibial segment of a prosthetic knee. The proximal connector 21 may be a standard prosthetics connection, such as a prosthetics pyramid adaptor receiver. The proximal connector 21 may be attached to the tibia proximal end portion 22 or unitarily formed with the tibia proximal end portion 22. The shaft 24 extends between the tibia proximal end portion 22 and the tibia distal end portion 26 and may be made in a variety of lengths to accommodate the anatomy of a user and/or other prosthetics of the user. For example, the shaft 24 may be longer for taller users and the shaft 24 may be shorter for shorter users.

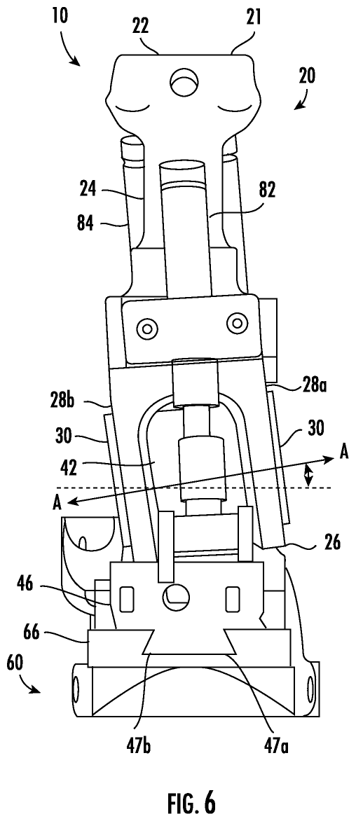

The tibia distal end portion 26 rotatably couples the tibia section 20 to the ankle section 40. The tibia distal end portion 26 includes a pair of arms 28a and 28b. The arms 28a, 28b are spaced apart from each other to receive the ankle section 40. The arms 28a, 28b each define an opening 29a, 29b and an ankle axis A-A. The openings 29a, 29b are aligned with each other and may house bearings 30 or bushings to aid relative rotation of the tibia section 20 and the ankle section 40. As best shown in FIG. 6, the arms 28a, 28b are angled relative to the transverse plane of the foot section 60 in the range of 4 degrees to 12 degrees, e.g., 8 degrees, so that the ankle axis A-A matches the axis of rotation of a biological ankle joint. In some embodiments, the angle of the ankle axis A-A with respect to the transverse plane of the ankle section 40 may be adjusted to match the ankle axis of the contralateral biological ankle of the user.

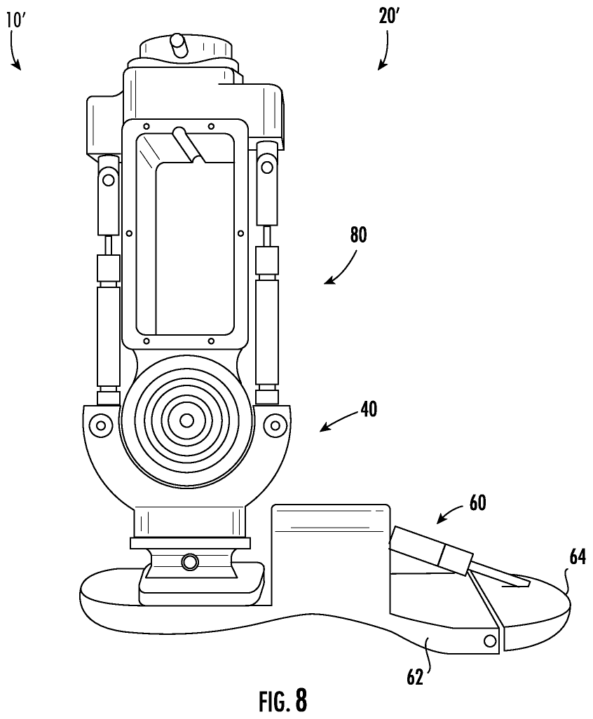

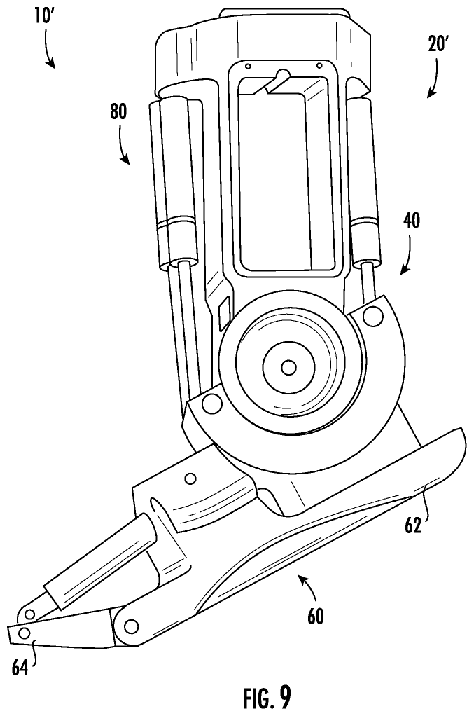

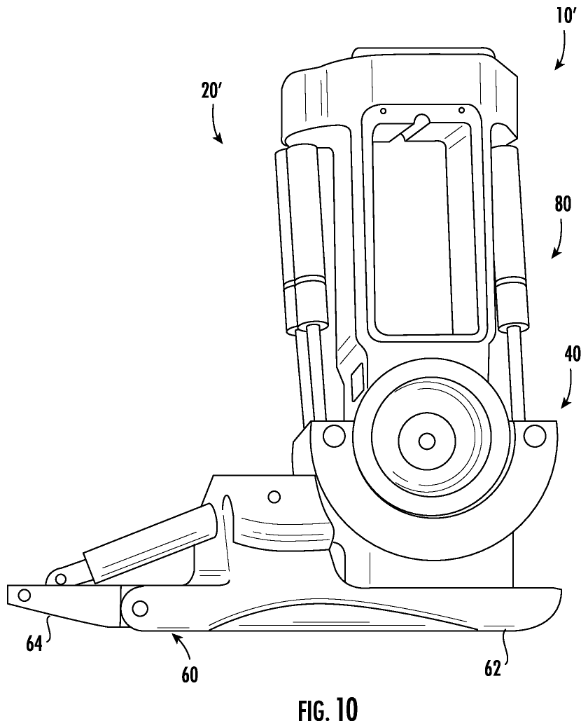

As a user moves through the gait cycle, the prosthetic ankle 10 moves between a neutral position (FIG. 8) in which the tibia section 20 is substantially perpendicular to the foot section 60, a plantar-flexion position (FIG. 9) in which the angle between the anterior side of the tibia section 20 and the superior side of the foot section 60 increases to be greater than 90-degrees, and a dorsi-flexion position (FIG. 10) in which the angle between the anterior side of the skeletonized tibia section 20′ and the superior side of the foot section 60 decreases to be less than 90-degrees. The anatomically correct axes allow the anatomically aligned prosthetic ankle 10 to more closely emulate the motion of an anatomically correct ankle or foot during the gait cycle.

Returning to FIGS. 4-7, the ankle section 40 rotatably couples to the tibia section 20 between the pair of arms 28a and 28b. The ankle section 40 includes an ankle projection 42 and an ankle distal end portion 44. The ankle projection 42 is received between the arms 28a, 28b and rotates relative to the tibia section 20 about the ankle axis A-A. The ankle projection 42 is angled relative to the transverse plane of the foot section 60 to align with the arms 28a, 28b, e.g., 8 degrees, so as to rotatably couple with the tibia section 20. The ankle projection 42 may be angled relative to the transverse plane of the ankle section 40 at an angle in a range of 4 degrees to 12 degrees, e.g., 8 degrees, in the frontal plane of the ankle section 40.

The ankle distal end portion 44 includes a coupling device 46 to connect the foot section 60 to the prosthetic ankle 10. As shown in FIG. 6, the coupling device 46 may be the male half 47a of a dove tail joint 47. The dove tail joint 47 may allow for sliding adjustment of the foot section 60 relative to the ankle section 40 in the anterior or the posterior direction. In some embodiments, the coupling device 46 may be a standard connection, such as a prosthetics pyramid adaptor.

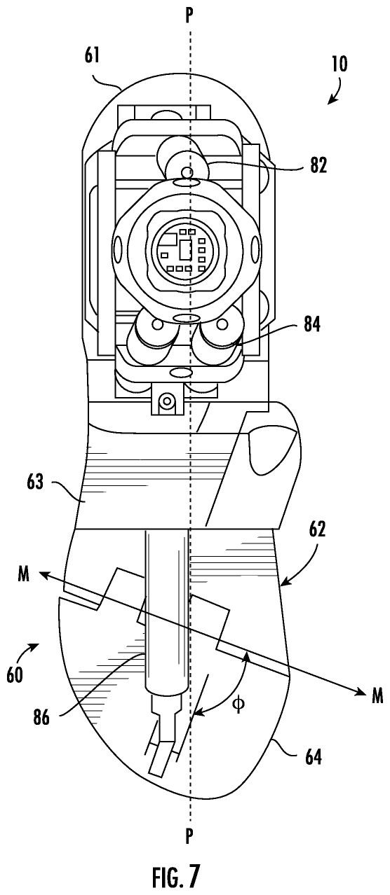

With particular reference to FIG. 7, the foot section 60 includes a foot base 62 and toe portion 64. The foot base 62 has a heel segment 61 and a mid-foot segment 63. The heel segment 61 includes a foot connector 66 corresponding to the coupling device 46 of the ankle distal end portion 44. In some embodiments, the foot connector 66 is the female half 47b of the dove tail joint 47 which allows the foot section 60 to be adjusted in the anterior or the posterior direction. The mid-foot segment 63 extends between the foot connector 66 and the toe portion 64.

The toe portion 64 is pivotally coupled to the foot base 62. The toe portion 64 is pivotable relative to the foot base 62 about a toe axis M-M between a toe neutral position (FIG. 8) and a toe flexion position (FIG. 9). In the toe neutral position, the inferior side of the toe portion 64 is substantially coplanar with the inferior side of the foot base 62. In the toe flexion position, the inferior side of the toe portion 64 pivots out of coplanarity with the inferior side of the foot base 62 such that the inferior side of the toe portion 64 is askew from the inferior side of the foot base 62. The inferior side of the toe portion 64 and the inferior side of the foot base 62 may be parallel with the transverse plane of the foot section 60 in the toe neutral position. In the toe flexion position, the inferior side of the toe portion 64 or the inferior side of the foot base 62 may be parallel with the transverse plane of the foot section 60. The toe portion 64 may pivot independently from movement or rotation of the tibia section 20 and the ankle section 40. For example, the prosthetic ankle 10 may be in the dorsi-flexion position and the toe portion 64 may be in the toe flexion position, e.g., when a user navigates an incline plane. Conversely, the prosthetic ankle 10 may be in the plantar-flexion position and the toe portion 64 may be in the toe neutral position, e.g., when a user navigates a decline plane such as a wheelchair ramp.

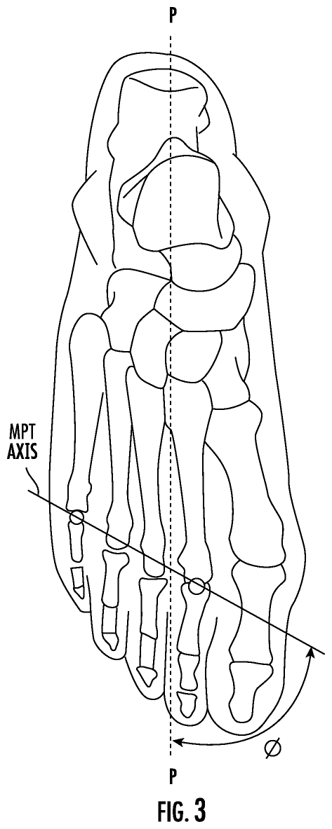

The toe axis M-M is angled relative to the sagittal plane of the foot section 60, or the line of progression P-P, along the midline of the foot section 60. The line of progression P-P is parallel to the medial plane of the body. The line of progression P-P defines the direction of locomotion of the user. The toe angle φ from the line of progression is such that the toe axis M-M corresponds to a biological metatarsophalangeal joint axis (hereinafter “the MPT axis”). The MPT axis is defined as the line extending between the MPT joint on the second phalange and the fifth phalange, as shown in FIG. 3. The toe angle φ is the angle between the MPT axis and the line of progression P-P in the transverse plane of the foot section 160 which is in the range of 50 degrees to 80 degrees, e.g., 70 degrees.

Continuing to refer to FIGS. 4-7, the flexion mechanism 80 includes a plurality of flexion members to passively assist movement of the tibia section 20, the ankle section 40, and the foot section 60 relative to each other and, thus, movement of the prosthetic ankle 10 between the neutral position, the dorsi-flexion position, and the plantar-flexion position. The flexion mechanism 80 includes a dorsi-flexion member 82, a plantar-flexion member 84, and a toe flexion member 86. The flexion members 82, 84, 86 may be hydropneumatic, hydraulic, or pneumatic dampers or springs. The flexion members 82, 84, 86 may be adjusted or selected to increase or decrease an assisting force exerted by a respective flexion member 82, 84, 86. For example, the toe flexion member 86 may be adjusted or selected to increase or decrease a push-off force transferred to the ground through the toe portion 64 during the gait cycle of the user.

The dorsi-flexion member 82 secures to the posterior side of the prosthetic ankle 10 and attaches to the tibia section 20 and to the ankle section 40. The dorsi-flexion member 82 urges rotation of tibia section 20 and the ankle section 40 toward the neutral position and resists rotation toward the plantar-flexion position. The plantar-flexion member 84 secures to the anterior side of the prosthetic ankle 10 and attaches to the tibia section 20 and to the ankle section 40. The plantar-flexion member 84 urges rotation of the tibia section 20 and the ankle section 40 toward the neutral position and resists rotation toward the dorsi-flexion position. The toe flexion member 86 secures to foot section 60 and attaches to the foot base 62 along the mid-foot segment 63 and attaches to the superior side of the toe portion 64. The toe flexion member 86 resists pivoting of the toe portion 64 toward the toe flexion position and urges the toe portion 64 to pivot toward the neutral position from the toe flexion position. Locomotion of the user through the gait cycle causes the tibia section 20, the ankle section 40, and the toe portion 80 to move relative to each other and the flexion members 82, 84, 86 to actuate. During the swing phase of the gait cycle the flexion members 82, 84, 86 return the prosthetic ankle 10 to the neutral position, thereby resetting the prosthetic ankle 10 for the user to take the next step in the gait cycle.

Referring to FIGS. 8-10, a skeletonized prosthetic ankle 10′ is provided in accordance with embodiments of the present disclosure. The skeletonized prosthetic ankle 10′ includes a skeletonized tibia section 20′. A skeletonized tibia section 20′ may reduce the overall weight of the skeletonized prosthetic ankle 10′ when compared to the prosthetic ankle 10. In some embodiments, the skeletonized tibia section 20′ may be monolithically formed, e.g., machined or formed from a single piece of material. In certain embodiments, the skeletonized tibia section 20′ may be assembled from a plurality of component parts, e.g., a plurality of machined plates fixed together to form the skeletonized tibia section 20′. Assembling the skeletonized tibia section 20′ from a plurality of component parts may decrease manufacturing complexities and costs.

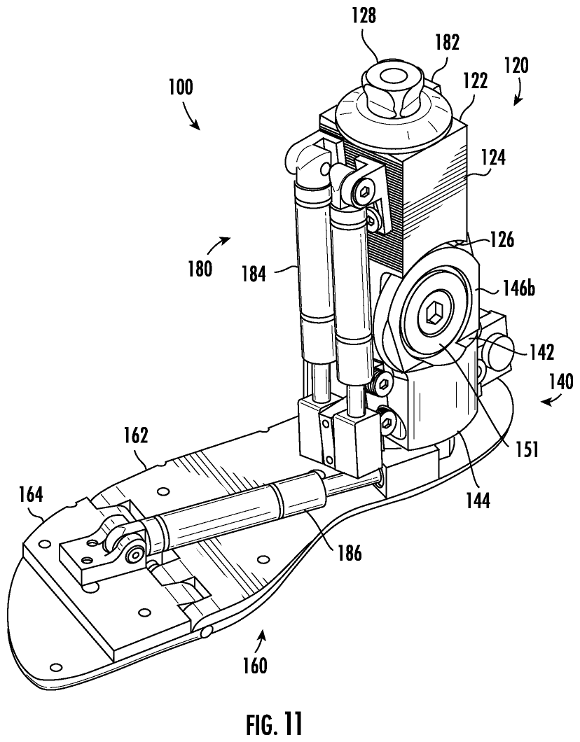

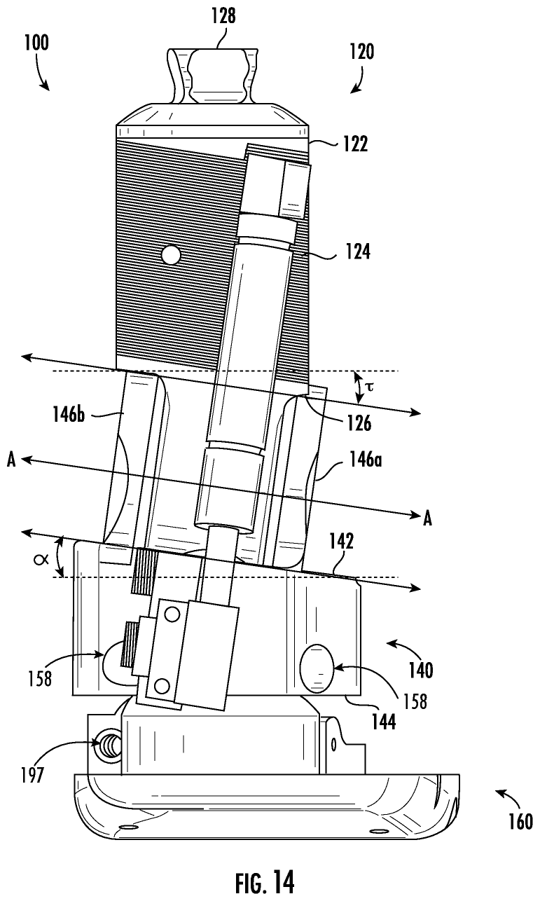

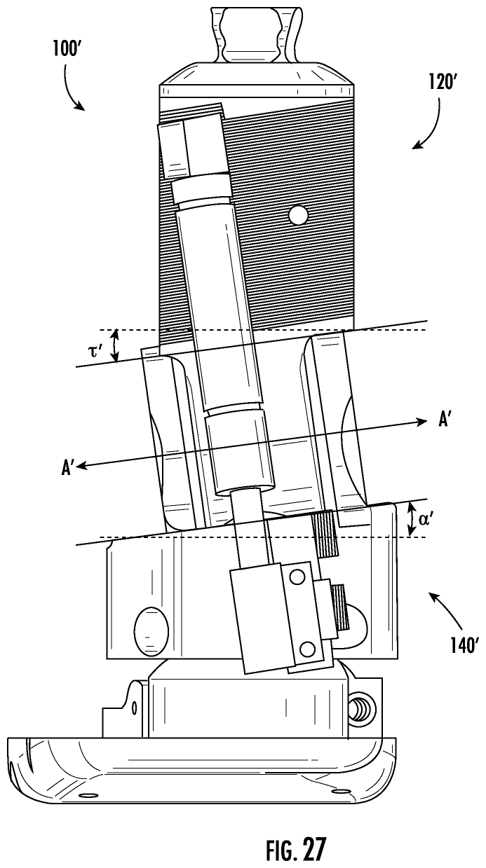

Now referring to FIGS. 11-14, another anatomically aligned prosthetic ankle 100 is provided in accordance with the present disclosure. The prosthetic ankle 100 includes a tibia section 120, an ankle section 140, and a foot section 160. The tibia section 120, the ankle section 140, and the foot section 160 move relative to each other about anatomically correct axes that correspond to the rotational axes of a biological ankle, foot, or knee. As the user moves through the gait cycle, the prosthetic ankle 100 moves between the neutral position, the plantar-flexion position, and the dorsi-flexion position passively assisted by a flexion mechanism 180. The anatomically correct axes allow the prosthetic ankle 100 to more closely emulate the motion of a biological limb during locomotion. Components of the prosthetic ankle 100 are universal and may be used to assemble either a right prosthetic ankle 100 or left prosthetic ankle 100′, as shown in FIG. 14 and FIG. 27 respectively. In addition, some elements of the prosthetic ankle 100 are similar to elements of the prosthetic ankle 10 detailed above with like elements represented with a preceding “1” before the previous label.

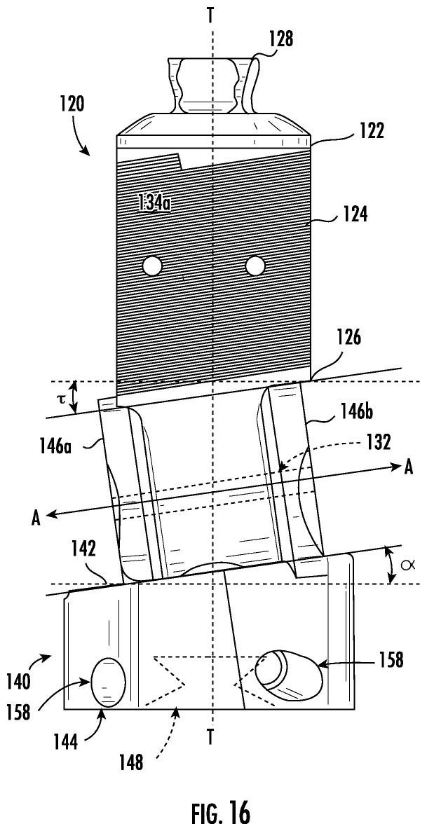

With particular reference to FIGS. 14-17, the tibia section 120 includes a tibia proximal end portion 122, a shaft 124, a tibia distal end portion 126, a proximal connector 128, and a tibia projection 130. The proximal connector 128 connects the prosthetic ankle 100 to a user. The proximal connector 128 may secure to a prosthetic socket (not shown) which receives the terminal end of a residual limb of a user, e.g., when the limb is amputated below the knee. In some embodiments, the proximal connector 128 secures to a prosthetic knee (not shown), e.g., when the limb is amputated above the knee. The proximal connector 128 is attached the tibia proximal end portion 122 and may be a standard prosthetics connection, such as a prosthetics pyramid adaptor. The proximal connector 128 may be attached to the tibia section 120 such that the proximal connector 128 defines a knee axis K-K angled relative to the frontal plane, or to an ankle axis A-A described below, in the transverse plane a knee angle ϰ in the range of 4 degrees to 12 degrees, e.g., 8 degrees. Such attachment of the proximal connector 128 to the tibia section 120 allows for alignment of the prosthetic ankle 100 and the knee of a user, either biological or prosthetic, to better match the alignment of the rotational axes between a biological knee and ankle. In embodiments, the proximal connector 128 is adjustable such that the knee axis K-K may be adjusted to match the alignment of the contralateral biological knee of a user.

The shaft 124 extends between the tibia proximal end portion 122 and the tibia distal end portion 126 and defines a tibial axis T-T extending vertically in the sagittal plane of the tibia section 120 and orthogonally to the transverse plane of the tibia section 120. The shaft 124 may be made in a variety of lengths to accommodate the anatomy of a user. The shaft 124 may be longer for taller users and may be shorter for shorter users. The tibia distal end portion 126 is angled relative to the transverse plane of the tibia section 120 at a tibia angle τ in the frontal plane of the tibia section 120. The tibia angle τ is in the range of 4 degrees to 12 degrees, e.g., 8 degrees. The tibia projection 130 extends perpendicularly from the tibia distal end portion 126 so as to be angled relative to the tibial axis T-T at the tibia angle τ. The tibia projection 130 defines a hole 132 extending in the lateral direction through the tibia projection 130 with the central axis of the hole 132 perpendicular to the medial side and the lateral side of the tibia projection 130 so as to match the tibia angle τ.

The anterior side and the posterior side of the shaft 124 may have a first half 134a of a cooperating adjustment mechanism 134 to allow for adjustment of the flexion mechanism 180. The cooperating adjustment mechanism 134 may be a set of teeth configured to mate with an opposing set of teeth. The cooperating adjustment mechanism 134 is formed so as to allow the universality of the tibia section 120. For example, where the cooperating adjustment mechanism 134 is formed as a set of teeth, the teeth may be biased relative to the tibial axis T-T to match the tibia angle τ to allow the tibia section 120 to be used in a right prosthetic ankle 100 or a left prosthetic ankle 100′ by rotating the tibia section 120 180-degrees about the tibial axis T-T.

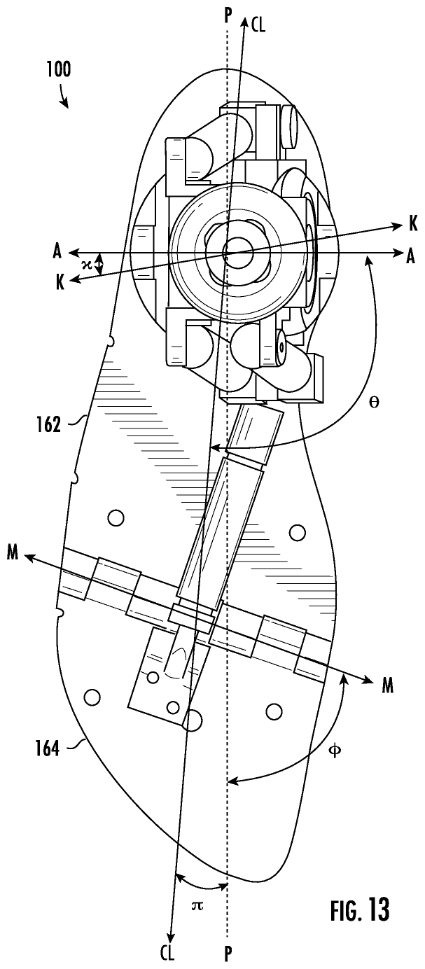

Continuing to refer to FIGS. 14-17, the ankle section 140 rotatably couples with the tibia projection 130 such that the ankle section 140 and the tibia section 120 rotate relative to each other about an ankle axis A-A. The ankle axis A-A is defined such that it corresponds to the rotational axis of a biological ankle. The ankle section 140 includes an ankle proximal end 142, an ankle distal end portion 144, and a pair of arms 146a, 146b. The ankle proximal end 142 is angled relative to the transverse plane of the ankle section 140 in the frontal plane of the ankle section 140 at an ankle angle α in the range of 4 degrees to 12 degree that cooperates with the tibia angle τ, e.g., 8 degrees. The ankle angle α is a non-zero angle and is away from parallel to the transverse plane of the ankle section 140 in the frontal plane. In some embodiments, the ankle angle α is adjustable such that the ankle axis A-A may be adjusted to match the angle of the contralateral, biological ankle of the user. The pair of arms 146a, 146b are spaced apart from each other and extend perpendicularly from the ankle proximal end 142 in the proximal direction. The pair of arms 146a, 146b each define a passage 150a and 150b, respectively, that house a bearing 151 to aid in relative rotation of the ankle section 140 and the tibia section 120. In embodiments, the bearings 151 may be replaced with a bushing. The pair of arms 146a, 146b define the ankle axis A-A extending through the passages 150a, 150b. The ankle axis A-A extends through the pair of arms 146a, 146b parallel to the ankle proximal end 142. Thus, the ankle axis A-A is angled relative to the transverse plane and in the frontal plane of the ankle section 140 in range of 4 degrees to 12 degrees, e.g., 8 degrees, or to match the rotational axis of a biological ankle. In some embodiments, a foot center line CL-CL is off-set from the ankle axis A-A at a foot angle θ in a transverse plane of the foot section 160 (FIG. 13). The foot angle θ is in the range of 65 degrees to 100 degrees, e.g., 85 degrees, or greater than 100 degrees. In certain embodiments, the foot angle θ is adjustable to match the foot angle of the contralateral, biological foot of the user.

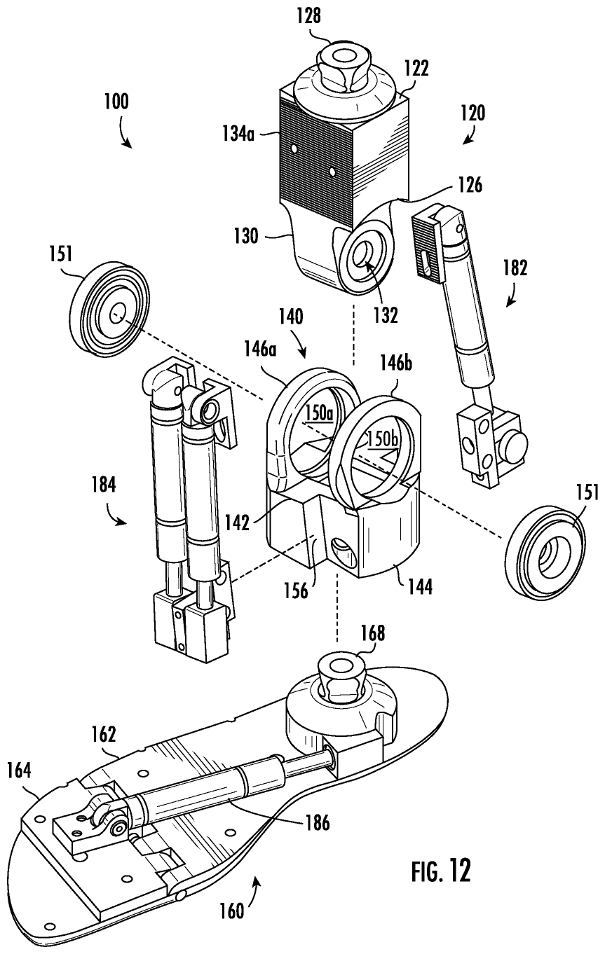

The ankle distal end portion 144 connects the foot section 160 to the ankle section 140. A socket 148 is defined by the ankle section 140 to receive a foot connector 168 (FIG. 12) and is positioned in the ankle distal end portion 144. A plurality of ports 158 may be disposed through the ankle section 140 and in communication with the socket 148 to allow fasteners, e.g., set screws, to secure the foot section 160 to the ankle section 140. The anterior side and the posterior side of the ankle section 140 includes a flexor mount surface 156 for securement of the flexion mechanism 180 to the ankle section 140. The flexor mount surface 156 is angled relative to the sagittal plane to be perpendicular to the ankle proximal end 142, e.g., 8 degrees, and to facilitate the universality of the prosthetic ankle 100.

The tibia section 120 and the ankle section 140 may be made of titanium or some other material having desirable strength to weight characteristics. For example, the tibia section 120 and the ankle section 140 may be made of aluminum, carbon fiber, titanium, steel, stainless steel, aluminum alloys, steel alloys, fiberglass, or other suitable materials. In some embodiments, the tibia section 120 and the ankle section 140 may be made of steel to reduce material costs.

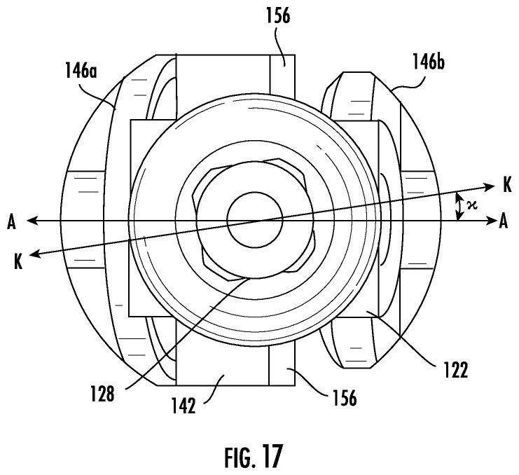

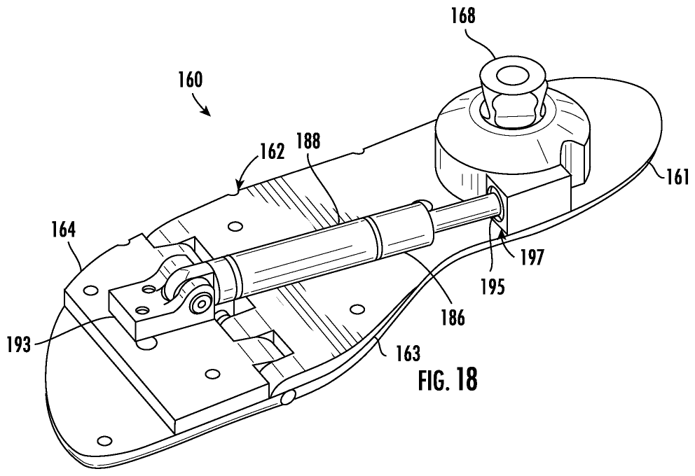

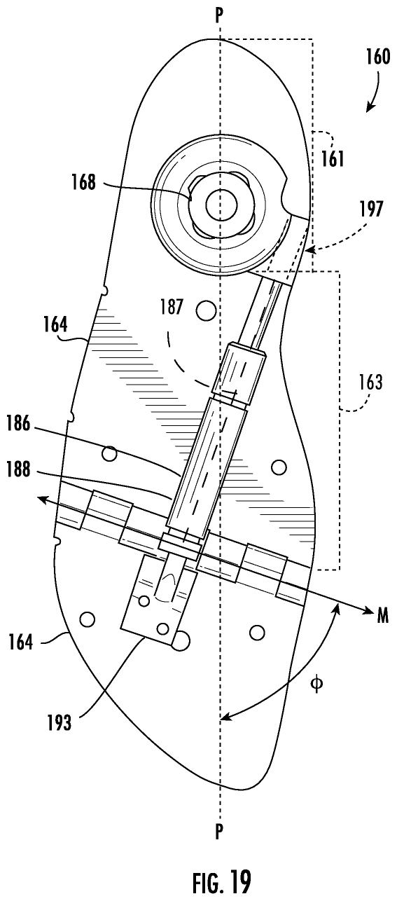

Now referring to FIGS. 18 and 19, the foot section 160 includes a base foot 162 and a toe portion 164. The base foot 162 has a heel segment 161 and a mid-foot segment 163. The heel segment 161 includes a foot connector 168 that couples the foot section 160 to the ankle section 140 and spans from the posterior most point of the base foot 162 to the anterior side of the foot connector 168. In some embodiments, the foot connector 168 is a standard prosthetics connector such as a prosthetics pyramid adaptor. The mid-foot segment 163 extends from the foot connector 168 in the anterior direction until the base foot 162 meets the toe portion 164. The foot section 160 and specifically, the mid-foot segment 163 may be made in various sizes to match the anatomy of the user. For example, a larger use may require a larger foot section 160 and thus, a longer and/or wider mid-foot segment 163 to aid in balance while standing or in locomotion. The foot section 160 has a foot centerline CL-CL that bisects the foot section 160 longitudinally. The foot centerline CL-CL may be off-set from the line of progression P-P at a non-zero progression angle π in a transverse plane of the foot section 160. The progression angle π may be in the range of 4 degrees to 12 degrees, e.g., 8 degrees. In some embodiments, the foot centerline CL-CL is parallel or coplanar with the line of progression P-P.

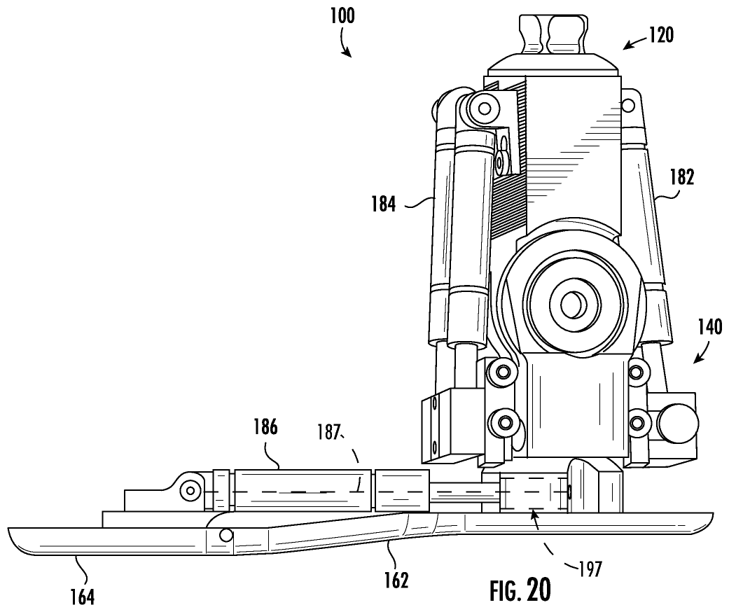

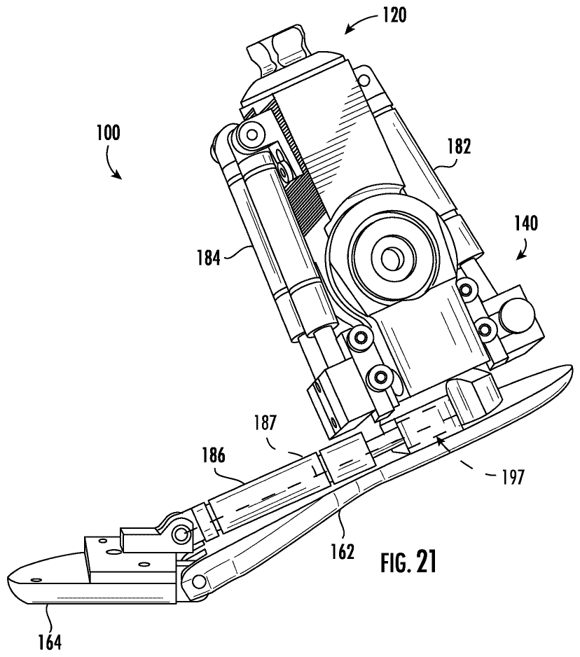

With additional reference to FIGS. 20 and 21, the toe portion 164 pivotally couples to the base foot 162 about a toe pivot axis M-M and is pivotable between a toe neutral position (FIG. 20) and a toe flexion position (FIG. 21). The toe pivot axis M-M is angled relative to the line of progression P-P of the foot section 160 at a toe angle φ in the transverse plane of the foot section 160 in the range of 50 degrees to 80 degrees, e.g., 70 degrees. The toe angle φ is selected to correspond with the angle of the MTP joint axis of a biological foot. In some embodiments, the toe angle φ is adjustable to match the angle of the MPT joint axis of the contralateral, biological foot of the user.

In the toe neutral position, the inferior side of the base foot 162 and the inferior side of the toe portion 164 are substantially coplanar with one another as shown in FIG. 20. In the toe flexion position the inferior side of the base foot 162 and the inferior side of the toe portion 164 pivot out of coplanarity with each other such that the inferior side of the toe portion 164 is askew from the inferior side of the base foot 162. The toe portion 164 may pivot independently of the relative rotation of the tibia section 120 and the ankle section 140. As shown in FIG. 21, the toe portion 164 may pivot while the tibia section 120 remains substantially perpendicular to the base foot 162, e.g., the prosthetic ankle 100 may remain in the neutral position. The prosthetic ankle 100 may be in the dorsi-flexion position and the toe portion 164 may be in the toe flexion position, e.g., when a user navigates an incline plane. Conversely, the prosthetic ankle 100 may be in the plantar-flexion position and the toe portion 164 may be in the toe neutral position, e.g., when a user navigates a decline plane such as a wheelchair ramp.

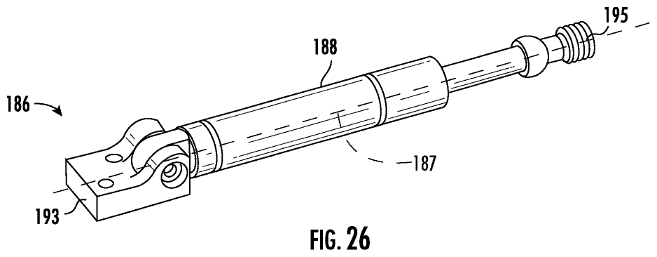

Referring to FIGS. 22-26, the flexion mechanism 180 aids the user in locomotion and provides passive assistance in moving the tibia section 120, the ankle section 140, and the foot section 160 relative to each other. For example, as the user moves through the gait cycle the flexion mechanism 180 may transfer force to the ground to provide a push-off force, e.g., through the toe portion 164. As the user raises the prosthetic ankle 100 off the ground during the swing phase of the gait cycle the flexion mechanism 180 returns the tibia section 120, the ankle section 140, and the toe portion 164 to their respective neutral positions. The flexion mechanism 180 includes a dorsi-flexion member 182, a plantar-flexion member 184, and a toe flexion member 186. The flexion members 182, 184, 186 may be hydropneumatics, hydraulic, or pneumatic springs and/or dampers. The flexion members 182, 184, 186 may be adjustable to increase or decrease the assisting force of their respective actions. In some embodiments, the assisting force of the flexion members 182, 184, 186 may be considered in equilibrium with each other such that the prosthetic ankle 100 is urged toward the neutral position and the toe portion 164 is urged toward the toe neutral position.

Each of the dorsi-flexion member 182 and the plantar-flexion member 184 include at least one piston 188, a rocker 190, a piston mount 192, and a rocker mount 194. The piston 188 may be a hydropneumatics damper. The piston 188 of the dorsi-flexion member 182 and the plantar-flexion member 184 provides the assisting force aiding in relative rotation between the tibia section 120 and the ankle section 140. In some embodiments, the piston 188 may be constructed as a strut, e.g., a coil spring disposed about an elongate rod. The piston 188 is swivelably and adjustably secured to the tibia section 120 by the piston mount 192 and is swivelably secured to the ankle section 140 by the rocker mount 194. The piston 188 is adjustably secured to the tibia section 120 such that the piston mount 192 may secure closer to the tibia proximal end portion 122 or the tibia distal end portion 126. Securing the piston 188 closer to the tibia proximal end portion 122 or the tibia distal end portion 126 may increase or decrease the assisting force applied by the piston 188. The piston 188 is swivelably secured to the tibia section 120 and the ankle section 140 such that the dorsi-flexion member 182 and the plantar-flexion member 184 swivel in concert with the tibia section 120 and the ankle section 140 as the prosthetic foot 100 moves between the plantar-flexion position and the dorsi-flexion position. The swivel action of the securement between the piston 188, the tibia section 120, and the ankle section 140 may reduce the chance the dorsi-flexion member 182, the plantar-flexion member 184, the tibia section 120, and the ankle section 140 of binding with each other.

The piston mount 192 may include the second half 134b of the cooperating adjustment mechanism 134. In some embodiments, the second half 134b of the cooperating adjustment mechanism 134 is a plurality of teeth that interlock with the first half 134a of the cooperating adjustment mechanism 134. In some embodiments, the piston mount 192 is fixedly secured to the tibia section 120 and the cooperating adjustment mechanism 134 is omitted. The rocker mount 194 includes a rocker saddle 196 and a rocker cap 198. The rocker saddle 196 couples to the ankle section 140 along the flexor mount surface 156 and the rocker cap 198 attaches to the rocker saddle 196 with a post 191 of the rocker 190 received therebetween to swivelably secure the piston 188 to the ankle section 140.

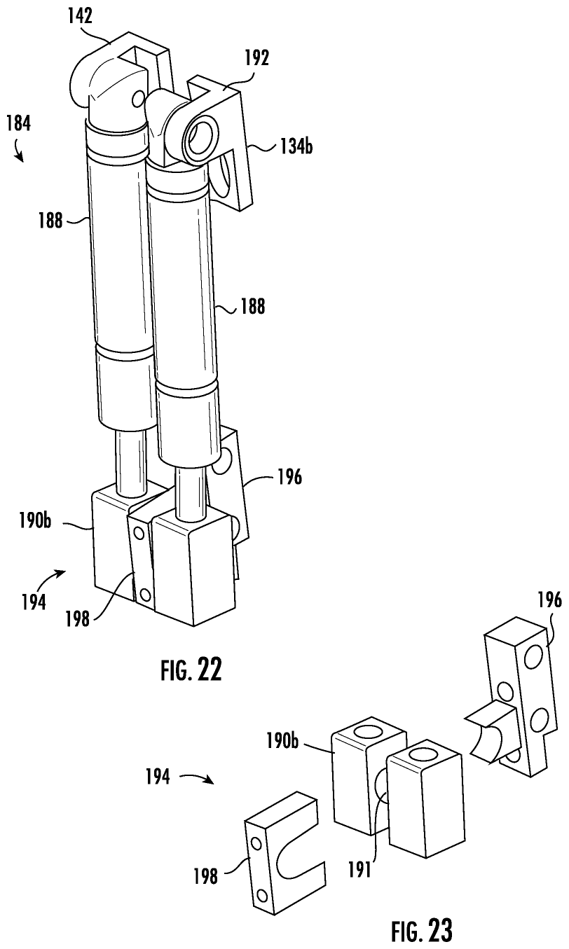

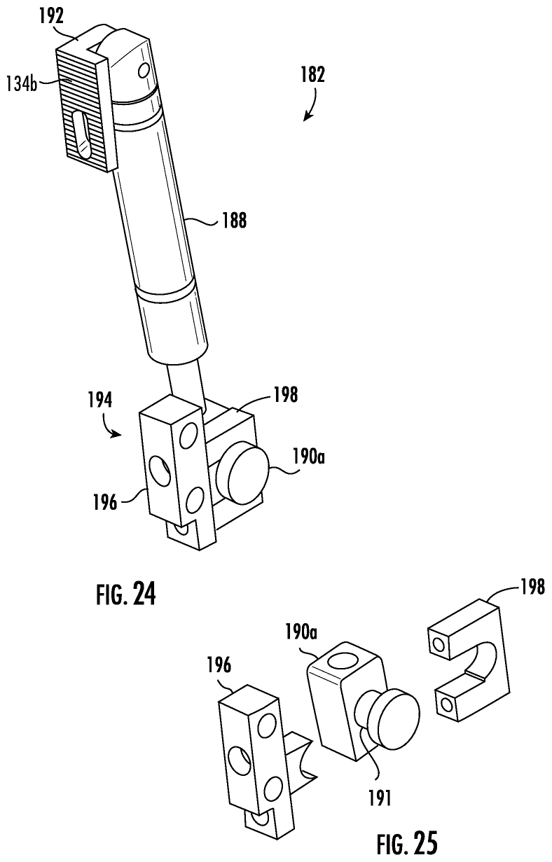

With particular reference to FIGS. 23 and 25, the rocker 190 may be formed as a single rocker 190a or a double rocker 190b. The single rocker 190a swivelably secures a dorsi-flexion member 182 or a plantar-flexion member 184 having only one piston 188 to the ankle section 140. The double rocker 190b swivelably secures a dorsi-flexion member 182 or a plantar-flexion member 184 having two pistons 188. The single rocker 190a and the double rocker 190b each include the post 191 that is received between the rocker saddle 196 and the rocker cap 198 and facilitates swivable securement of the dorsi-flexion member 182 or the plantar-flexion member 184. The dorsi-flexion member 182 and the plantar-flexion member 184 may each be assembled with either a single rocker 190a or a double rocker 190b and corresponding number of pistons 188.

The toe flexion member 186 includes a piston 188, a toe mount 193, and an adjustment device 195. The piston 188 of the toe flexion member 186 provides the assisting force aiding the toe portion 164 in pivoting relative to the base foot 162. The toe mount 193 secures the piston 188 to the toe portion 164 such that the toe flexion member 186 urges the toe portion 164 toward the neutral position and allows the toe portion 164 to pivot relative to the base foot 162 about the toe axis M-M. The piston 188 of the toe flexion member 186 is adjustably coupled to the foot connector 168. The foot connector 168 defines a piston receptacle 197 that receives the adjustment device 195. Rotation of the adjustment device 195 within the piston receptacle 197 may increase or decrease the assisting force exerted by the toe flexion member 186 on the toe portion 164 and, thus, the push-off force applied to the ground during locomotion. As the toe portion 164 pivots between the toe neutral position and the toe flexion position, a central axis 187 of the toe flexion member 186 may remain substantially parallel to the inferior side of the base foot 162, as shown in FIGS. 20 and 21. The central axis 187 may be substantially perpendicular to the toe axis M-M as shown in FIG. 19.

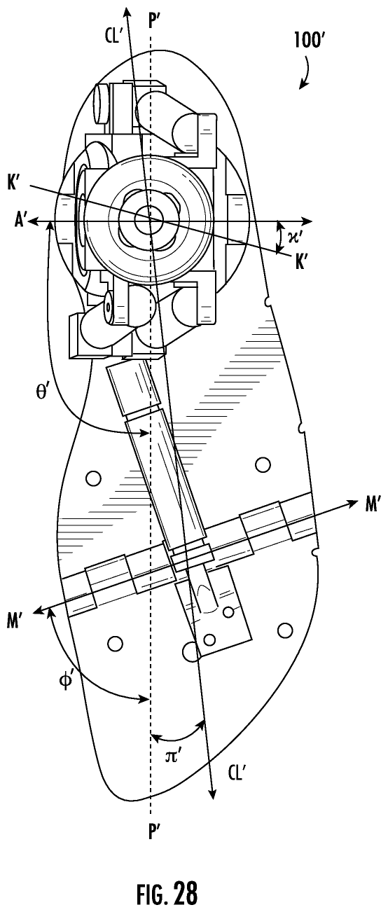

With reference to FIGS. 13, 14, 27, and 28, the prosthetic ankle 100 includes universal components. In other words, certain components of the prosthetic ankle 100 may be used in the assembly of either a right prosthetic ankle 100 or a left prosthetic ankle 100′. A left prosthetic ankle 100′ is shown with labels including prime notation to distinguish identical components and resultant axes in a left ankle configuration from the right prosthetic ankle 100 of FIGS. 11-26 shown in a right ankle configuration.

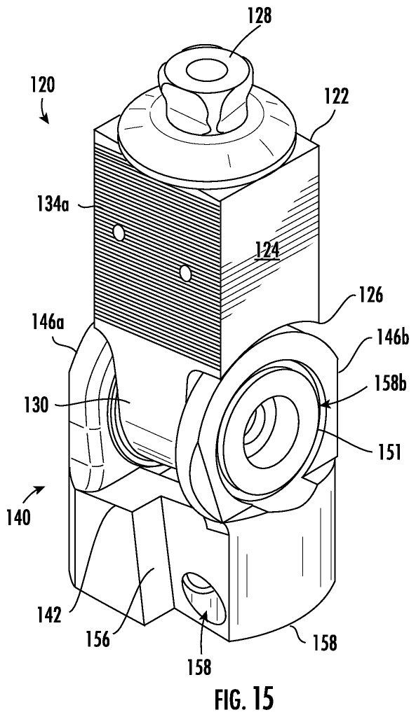

Specifically, the tibia section 120, the ankle section 140, the dorsi-flexion member 182, and the plantar-flexion member 184 are universal components. The tibia section 120, the ankle section 140, and the flexion mechanism 180 may be used to assemble to the respective left prosthetic ankle 100′. The tibia section 120 and the ankle section 140 may be switched to their left ankle configurations 120′ and 140′ by rotating them 180-degrees about their respective vertical axes. For example, the tibia section 120 and ankle section 140 may be configured as the left tibia section 120′ and the left ankle section 140′ by rotation 180 degrees about the tibial axis T-T (FIG. 16). The anatomically correct axes are preserved from the right configuration to the left configuration and are labeled with prime notation, e.g., the ankle axis A-A of the right prosthetic ankle 100 is labeled as the ankle axis A′-A′ of the left prosthetic ankle 100′.

Switching the dorsi-flexion member 182 and the plantar-flexion member 184 to their respective left ankle configurations may require swapping their respective positions as a result of rotating the tibia section 120 and the ankle section 140 180-degrees about the tibial axis T-T. In other words, the dorsi-flexion member 182 is un-secured from the anterior sided of the left tibia section 120′ and the left ankle section 140′ and re-secured to the posterior side of the left tibia section 120′ and the left ankle section 140′, and the plantar-flexion member 184 is un-secured from the posterior sided of the left tibia section 120′ and the left ankle section 140′ and re-secured to the anterior side of the left tibia section 120′ and the left ankle section 140′. In embodiments, where the dorsi-flexion member 182 and the plantar-flexion member 184 are both formed with either the single rocker 190a or the double rocker 190b the universal components can be switched to the left ankle configuration without disassembly of the dorsi-flexion member 182 and the plantar-flexion member 184 from the tibia section 120 and the ankle section 140 by rotation 180-degrees about the tibial axis T-T of the entire sub-assembly.

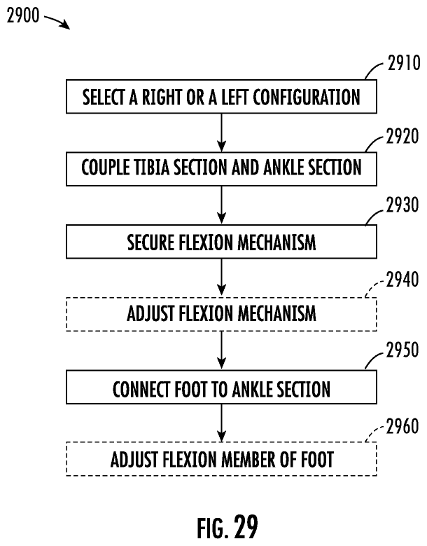

With reference to FIG. 29, a method 2900 of assembling a prosthetic ankle is described in accordance with the present disclosure with reference to the prosthetic ankle 100 of FIGS. 11-28

[symbol]

The method 2900 is used to assemble either a right prosthetic ankle 100 or a left prosthetic ankle 100′. In some embodiments, the method 2900 may be used to switch a right prosthetic ankle 100 to a left prosthetic ankle 100′ or vice versa. Once a right prosthetic ankle 100 or a left prosthetic ankle 100′ has been selected (Step 2910), the tibia section 120 is rotatably coupled to the ankle section 140 (Step 2920) with the appropriate orientation to define the desired ankle axis A-A or A′-A′. For example, in the assembly of a right prosthetic ankle 100, the tibia projection 130 is received between the arms 146a, 146b with the hole 132 aligned with the passages 150a, 150b such that the ankle axis A-A passes through the hole 132. The tibia section 120 and the ankle section 140 rotate relative to each other about the ankle axis A-A. The rotation of the tibia section 120 and the ankle section 140 may be aided by bearings 151. In some embodiments, the bearings 151 are substituted for bushings. When the tibia section 120 and the ankle section 140 are rotatably coupled, the knee axis K-K, defined by the proximal connector 128, is offset from the ankle axis A-A at the knee angle ϰ in the range of 4 degrees to 12 degrees, e.g., 8 degrees.

The flexion mechanism 180 is secured to the tibia section 120 and the ankle section 140 (Step 2930). Specifically, the dorsi-flexion member 182 is secured to the posterior sides of the tibia section 120 and the ankle section 140, and the plantar-flexion member 184 is secured to the anterior sides of the tibia section 120 and the ankle section 140. The rocker mount 194 of the dorsi-flexion member 182 and the plantar-flexion member 184 secure to a respective flexor mount surface 156 of the ankle section 140. The piston mount 192 of the dorsi-flexion member 182 and the plantar-flexion member 184 secure to the posterior side and the anterior side of the tibia section 120, respectively. The piston mount 192 may adjustably secure the dorsi-flexion member 182 and the plantar-flexion member 184 to the tibia section 120. The dorsi-flexion member 182 and the plantar-flexion member 184 may be adjusted by securing the piston mount 192 closer to the tibia proximal end portion 122 or the tibia distal end portion 126 (Step 2940). Adjustment of the dorsi-flexion member 182 and the plantar-flexor member 184 is facilitated by engagement of the second half 134b of the cooperating adjustment with the first half 134a of the cooperating adjustment mechanism 134. Securing the piston mount 192 closer to the tibia proximal end portion 122 or the tibia distal end portion 126 may increase or decrease the assisting force exerted by the respective flexion member 182, 184 of the flexion mechanism 180.

Either the right foot section 160 or the left foot section 160′ is connected to the ankle section 140 by the foot connector 168 (Step 2950). The foot connector 168 is received in the socket 148 to connect the foot section 160 to the ankle section 140. In embodiments where the foot connector 168 is a prosthetic pyramid connector, the foot connector 168 may be fastened by a plurality of set screws disposed within ports 158 defined in the ankle section 140. The assisting force exerted by the toe flexion member 186 may be increased or decreased by rotating the adjustment device 195 clockwise or counter-clockwise (Step 2960).

Although the method steps are described in a specific order, it should be understood that other steps may be performed in between described steps, described steps may be adjusted so that they occur at slightly different times, or the described steps may occur in any order unless otherwise specified.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A prosthetic limb having transverse planes parallel to a horizontal plane of a user and sagittal planes parallel to a medial plane of the user, the prosthetic limb comprising:
a tibia section;
an ankle section defining an ankle axis, the ankle section rotatably coupled to the tibia section and configured to rotate relative to the tibia section about the ankle axis; and
a foot section secured to the ankle section, the foot section having a foot sagittal plane defining a line of progression of the foot section, the foot section comprising a base foot and a toe portion pivotally coupled to the base foot, the toe portion configured to pivot about a toe axis relative to the base foot, the toe axis angled relative to the line of progression at a toe angle in a transverse plane of the foot section, the toe angle being less than 90 degrees.

2. The prosthetic limb according to claim 1, wherein the toe angle is between 50 degrees and 80 degrees.

3. The prosthetic limb according to claim 1, wherein the toe angle is 70 degrees.

4. The prosthetic limb according to claim 1, wherein the tibia section and the ankle section are configured to be the tibia section and the ankle section of a right prosthetic limb or a left prosthetic limb.

5. The prosthetic limb according to claim 1, wherein the tibia section and the ankle section are configured to switch between the tibia section and the ankle section of a right prosthetic limb and a left prosthetic limb by rotating the tibia section and the ankle section 180-degrees about a central longitudinal axis thereof.

6. The prosthetic limb according to claim 1, wherein the foot section has a foot center line bisecting the foot section longitudinally, the foot center line off-set relative to the ankle axis at a foot angle in the transverse plane of the foot section, the foot angle in a range of 50 degrees to 100 degrees.

7. The prosthetic limb according to claim 1, comprising a toe flexion member secured to the base foot and the toe portion, the toe flexion member configured to assist pivoting of the toe portion relative to the base foot.

8. The prosthetic limb according to claim 7, wherein the toe flexion member comprises an adjustment device configured to increase or decrease an assisting force applied by the toe flexion member to assist pivoting of the toe portion relative to the base foot.

9. The prosthetic limb according to claim 8, wherein the toe flexion member has a first end secured the base foot and a second end, opposite the first end, secured to the toe portion.

10. The prosthetic limb according to claim 9, wherein the toe flexion member defines a central axis and is secured to the base foot and the toe portion such that the central axis is substantially perpendicular to the toe axis.

11. The prosthetic limb according to claim 7, wherein the toe flexion member comprises a hydropneumatics damper.

12. The prosthetic limb according to claim 1, wherein the toe portion has an inferior side and the base foot has an inferior side, and the toe portion is configured to pivot relative to the base foot between a toe neutral position in which the inferior side of the toe portion is substantially coplanar to the inferior side of the base foot and a toe flexion position in which the inferior side of the toe portion is pivoted away from coplanarity with the inferior side of the base foot.

13. The prosthetic limb according to claim 12, comprising a toe flexion member configured to apply a push-off force to a ground surface as the toe portion pivots from the toe flexion position to the toe neutral position to assist a user in locomotion.

14. The prosthetic limb according to claim 13, wherein the toe flexion member comprises an adjustment device configured to increase or decrease the push-off force applied by the toe flexion member.

15. The prosthetic limb according to claim 13, wherein the toe flexion member defines a central axis and is secured to the base foot and the toe portion such that the central axis is substantially parallel to the inferior side of the base foot when the toe portion is in the toe neutral position and when the toe portion is in the toe flexion position.

16. The prosthetic limb according to claim 1, comprising a foot connector attached to a superior side of the base foot configured to operably secure the prosthetic limb to a user.

17. A prosthetic limb having transverse planes parallel to a horizontal plane of a user, frontal planes parallel to a coronal plane of the user, and sagittal planes parallel to a medial plane of the user, the prosthetic limb comprising:
a tibia section;
an ankle section defining an ankle axis that is angled relative to the transverse plane at a non-zero ankle angle in a frontal plane of the ankle section, the ankle section rotatably coupled to the tibia section and configured to rotate relative to the tibia section about the ankle axis; and
a foot section secured to the ankle section, the foot section having a foot center line and a foot sagittal plane, the foot center line longitudinally bisecting the foot section, the foot sagittal plane defining a line of progression of the foot section, the foot center line off-set relative to the line of progression at a non-zero progression angle in a transverse plane of the foot section, the foot section comprising a base foot and a toe portion.

18. The prosthetic limb according to claim 17, wherein the toe portion is pivotally coupled to the base foot and is configured to pivot relative to the base foot.

19. The prosthetic limb according to claim 17, wherein the progression angle is in the range of 4 degrees to 12 degrees.

20. The prosthetic limb according to claim 17, wherein the progression angle is 8 degrees.

21. The prosthetic limb according to claim 17, wherein the ankle angle is in the range of 4 degrees to 12 degrees.

22. The prosthetic limb according to claim 17, wherein the ankle angle is 8 degrees.

23. The prosthetic limb according to claim 17, wherein the tibia section has an anterior side and the foot section has a superior side, the ankle section and the tibia section being rotatable relative to each other about the ankle axis between a neutral position in which the anterior side of the tibia section is substantially perpendicular to the superior side of the foot section, a dorsi-flexion position in which an angle between the anterior side of the tibia section and the superior side of the foot section is decreased, and a plantar-flexion position in which the angle between the anterior side of the tibia section and the superior side of the foot section is increased.

24. The prosthetic limb according to claim 23, comprising a flexion mechanism configured to passively assist in relative rotation between the tibia section and the ankle section, and pivoting of the toe portion relative the base foot.

25. The prosthetic limb according to claim 24, wherein the flexion mechanism comprises a dorsi-flexion member configured to assist rotation of the tibia section and the ankle section between the neutral position and the dorsi-flexion position.

26. The prosthetic limb according to claim 24, wherein the flexion mechanism comprises a plantar-flexion member configured to assist rotation of the tibia section and the ankle section between the neutral position and the plantar-flexion position.

27. The prosthetic limb according to claim 24, wherein the flexion mechanism comprises a toe flexion member configured to assist pivoting of the toe portion relative to the base foot.

28. The prosthetic limb according to claim 17, comprising a flexion mechanism configured to assist relative rotation of the tibia section and the ankle section, and pivoting of the toe portion relative to the base foot.

29. The prosthetic limb according to claim 28, wherein the flexion mechanism is configured to assist relative rotation of the tibia section and the ankle section, and pivoting of the toe portion relative to the base foot independently of one another.

30. The prosthetic limb according to claim 17, wherein the tibia section and the ankle section are configured to be the tibia section and the ankle section of a right prosthetic limb or a left prosthetic limb.

31. A prosthetic limb having transverse planes parallel to a horizontal plane of a user and frontal planes parallel to a coronal plane of the user, the prosthetic limb comprising:
a tibia section; and
an ankle section rotatably coupled to the tibia section, the ankle section and the tibia section rotatable about an ankle axis that is angled at an ankle angle relative a transverse plane of the ankle section in a frontal plane of the ankle section, the ankle angle being a non-zero angle, the tibia section and the ankle section switchable between a left prosthetic limb configuration and a right prosthetic limb configuration by rotating the tibia section and the ankle section 180-degrees about a central longitudinal axis thereof.

32. The prosthetic limb according to claim 31, wherein the ankle angle is in the range of 4 degrees to 12 degrees.

33. The prosthetic limb according to claim 31, wherein the ankle angle is 8 degrees.

34. The prosthetic limb according to claim 31, comprising a dorsi-flexion member configured to assist relative rotation between the tibia section and the ankle section, the dorsi-flexion member switchable between a right prosthetic limb configuration and a left prosthetic limb configuration by securing the dorsi-flexion member to a posterior side of the respective right prosthetic limb configuration of the tibia section and the ankle section or the left prosthetic limb configuration of the tibia section and the ankle section.

35. The prosthetic limb according to claim 31, comprising a plantar-flexion member configured to assist relative rotation between the tibia section and the ankle section, the plantar-flexion member switchable between a right prosthetic limb configuration and a left prosthetic limb configuration by securing the plantar-flexion member to an anterior side of the respective right prosthetic limb configuration of the tibia section and the ankle section or the left prosthetic limb configuration of the tibia section and the ankle section.

36. A method of assembling a prosthetic limb having transverse planes parallel to a horizontal plane of a user, frontal planes parallel to a coronal plane of the user, and sagittal planes parallel to a medial plane of the user, the method comprising:
orientating a tibia section and an ankle section of the prosthetic limb in one of a right prosthetic limb configuration or a left prosthetic limb configuration, the tibia section and the ankle section rotatable relative to each other about an ankle axis, the ankle axis offset relative to the transverse plane at a non-zero ankle angle in a frontal plane of the ankle section; and
securing a right foot section or a left foot section to the ankle section based on the orientation of the tibia section and the ankle section.

37. The method according to claim 36, comprising securing a dorsi-flexion member and plantar-flexion member to the tibia section and the ankle section such that the dorsi-flexion member and the plantar-flexion member passively assist relative rotation between the tibia section and the ankle section.

38. The method according to claim 37, comprising adjusting the dorsi-flexion member or the plantar-flexion member to increase or decrease a force assisting relative rotation between the tibia section and the ankle section.

39. The method according to claim 36, adjusting a toe flexion member of the right foot section or the left foot section to increase or decrease a force assisting pivoting of a toe portion of the right foot section or of the left foot section.

40. The method according to claim 36, comprising switching the prosthetic limb to the other of the right prosthetic limb configuration or the left prosthetic limb configuration by rotating the tibia section and the ankle section 180-degrees about a central longitudinal axis thereof.

41. The method according to claim 40, further comprising:
removing a foot section from the ankle section;
reorientating the tibia section and the ankle section from one of the right prosthetic limb configuration or the left prosthetic limb configuration to the other of the right prosthetic limb configuration or the left prosthetic limb configuration; and
attaching another foot section matching the reorientated tibia section and ankle section.
